(19) United States
(12) Patent Application Publication
(10) Pub. No.: US 2009/0233966 A1
(43) Pub. Date: Sep. 17, 2009

(54) Analogues of the Azinomycins as Anti-Tumour Agents and as Prodrugs

(75) Inventors: Mark Searcey, Norfolk (GB); Laurence Hylton Patterson, West Yorkshire (GB); Klaus Pors, West Yorkshire (GB); Maxwell Casely-Hayford, Chatham (GB)

Correspondence Address: OLIFF & BERRIDGE, PLC, P.O. BOX 320850, ALEXANDRIA, VA, 22320-4850, US

(73) Assignee: School of Pharmacy, University of London, London (GB)

(21) Appl. No.: 11/886,505

(22) PCT Filed: Mar. 16, 2006
(86) PCT No.: PCT/GB2006/000941

§ 371 Date: May 22, 2008

(30) Foreign Application Priority Data
Mar. 18, 2005 (GB) 0505644.5

(51) Int. Cl. A61K 31/445; A61K 31/4523; C07D 211/06; C07D 405/12

(52) U.S. Cl. 514/319; 514/326; 546/206; 546/207

(57) ABSTRACT

Compounds of general formula (I) or a salt thereof in which R¹ is preferably an aromatic DNA binding subunit are oxidation-activated prodrugs. The compounds are expected to be converted into an epoxide at the alkene to which R² is attached by cytochrome P450, in particular CYPIBI, expressed at high levels in tumours. R³ preferably comprises a Nitrogen mustard to provide a prodrug which has 2 alkylating groups. The prodrugs are expected to be activated preferentially in tumour cells.

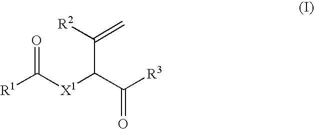

The present invention concerns aromatic oxidation-activated prodrugs, particularly anti-tumour prodrugs and those which are activated by the oxidation activities of the cytochrome P450 family of enzymes. The prodrugs may be alkylating agents having topoisomerase II inhibiting activities.

Many conventional cytotoxic drugs are known that can be used for therapeutic purposes. However, they typically suffer from the problem that they are generally cytotoxic and therefore may affect cells other than those that are required to be destroyed. This can be alleviated to some extent by the use of targeted drug delivery systems, for example direct injection to a site of tumourous tissue or, e.g. binding the cytotoxic agent to an antibody that specifically recognises an antigen displayed only on the cancer cell surface. Alternatively, electromagnetic radiation my be used to cause chemical alteration in an agent at a desired site such that it becomes cytotoxic. However, all of these techniques have, to a greater or lesser extent, certain limitations and disadvantages.

The azinomycins A and B are potent anti-tumour agents that bind to DNA by alkylation in the major groove and lead to cell death. However, they are relatively unstable, have poor availability from natural sources and are unlikely to proceed into the clinic.

These naturally occurring compounds, along with the truncated analogue A (see structure below), were first isolated from Streptomyces griseofuscus S42227 by Nagaoka et al in Japan (J. Antibiot. (Tokyo) 1986, 39, 1527-1532).

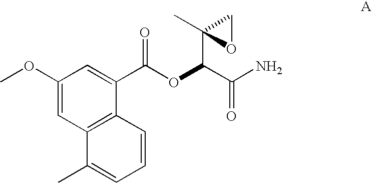

Armstrong in Tetrahedron Lett. 1991, 32, 3807-3810 later disclosed using mass and NMR special data, that the anti-tumour antibiotic carzinophilin, isolated in 1954 from Streptomyces sahachiror (Onda et al, J. Antibiot. 1969, 22, 42-44) was the same compound as natural product azinomycin B.

Shibuya in Tetrahedron Lett. 1983, 24, 1175-1178 describes the first synthetic studies of the azinomycins but these are inaccurate as they were based upon the erroneous structure of carzinophilin suggested by Lain et al in J. Am. Chem. Soc. 1982, 104, 3213-3214.

Truncated analogue A was first correctly synthesized by Shibuya et al in Tetrahedron Lett. 1987, 28, 2619-2622 where the commercially available diacetone D-glucose from the chiral pool was used in a lengthy multistep synthesis to stereospecifically generate the analogue A, of the structure shown above, with the same stereochemistry as the natural products.

The majority of other studies on the epoxide fragment of the azinomycins and on the synthesis of A have focused on the use of Sharpless asymmetric epoxidation. Direct efforts on synthesising enantiopure precursors are described by Konda et al in Chem. Pharmac. Bull 1994, 42, 285-288. Shipman et al in Chem. Soc. Perkin Trans. 1 1998, 1249-1255 further discuss a Sharpless asymmetric dihydroxylation/asymmetric epoxidation methodology to give the required S,S isomer in excellent yield.

Both Armstrong et al (J. Am. Chem. Soc. 1992, 114, 371-372) and Coleman et al (J. Org. Chem. 1992, 57, 5813-5815) have independently described synthetic routes to the aziridine core of Azinomycin A. The total synthesis proved more elusive and has only recently been described by Coleman et al (Angew. Chem. Int. Ed. 2001, 49 1736-1739). The key to the total synthesis was assembly of the backbone of the natural product, including the epoxide moiety, followed by the late stage introduction of the azabicyclic system through a Wadsworth-Horner-Emmons reaction.

The synthesis of the left-hand fragment of the azinomycins allowed the study of its interactions with DNA. Zang et al in Biochemistry 2000, 39, 14968-14975 present data to suggest that structure A intercalates with DNA via its naphthalene subunit and alkylates guanine residues at N7 with little, if any sequence selectivity. Shipman et al used these findings in structure-activity surveys to identify analogues of the natural products that might be useful as anti-tumour agents. (Bioorg. Med. Chem. Lett 2000, 10, 239-241). Replacement of the 3-methoxy-5-methylnaphthalene with a phenyl group (which would be expected to show little affinity for DNA through intercalation) effectively removed the biological potency of the epoxide in a variety of cell lines. In Chem. Commun. 2000, 2325-2326 Hortley et al study the DNA cross-linking activity of symmetrical dimers based upon the epoxide domain of the azinomycins. They demonstrated that an optimum linker length appeared to be 4 methylene groups and that the agents can cross-link DNA, and have potent cytotoxic activity, although none of the compounds had significantly greater activity than the non-crosslinking A.

The azinomycins appear to act by disruption of cellular DNA replication by interstrand crosslink formation. Lain et al in Can. J. Biochem. 1997, 55, 630-635 first noted the ability of azinomycin B to form covalent links between complementary strands of DNA. Fujiwara et al in Tetrahedron Lett. 1999, 40, 315-318 further suggest that the crosslinking occurs via an initial alkylation of the aziridine with the N7 of adenine followed by efficient crosslinking through a second reaction of the N7 of a guanine 2 bases away with the epoxide.

Casely-Hayford et al in Bioorganic and Med. Chem. Letters (2005) 15, 653-656, discuss the design and synthesis of a potentially therapeutically-viable azinomycin analogue B based upon A involving the coupling of a piperidine mustard to the acid chloride of the azinomycin chromophore.

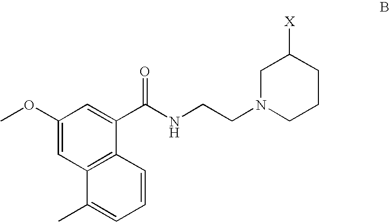

The authors conclude that monoalkylation is sufficient for biological activity and that crosslinking may even be detrimental.

The present invention relates to the first therapeutic use of a range of azinomycin analogues and their synthesis. The compounds incorporated herein are new. The present invention also relates to synthetic precursors of azinomycin analogues which do not have the epoxide or the aziridine ring of the natural products, and which are substantially inactive as DNA alkylating agents themselves.

It has been reported (Murray et al, 1997, Cancer Research, 57, 3026-3031 and WO-A-97 12246) that the enzyme CYP1B1, a member of the cytochrone P450 (CYP) family of xenobiotic metabolizing enzymes, is expressed at high frequency in a range of human cancers, including cancers of the breast, colon, lung, oesophagus, skin, lymph node, brain and testes, and that it is not detectable in normal tissue. This led to the conclusion that the expression of cytochrome P450 isoforms in tumour cells provides a molecular target for the development of new anti-tumour drugs that could be selectively activated by the CYP enzymes in tumour cells, although no drug examples were given. A number of other CYP isoforms have been shown to be expressed in various tumors. Many of the CYPs expressed in tumors are mentioned in Patterson, L H et al (1999) Anticancer Drug Des. 14(6)473-486.

In WO 02/067930A1 Searcey and Patterson describe various benz-indole and benzo-quinoline compounds as CYP-oxidisable prodrugs for tumour treatment. In WO 02/068412A1 they further describe pyrrolo-indole and pyrrolo-quinoline derivatives for use as CYP-oxidizable prodrugs and in WO 02/067937A1 indoline and tetrahydro-quinoline CYP-oxidisable prodrugs are described. All of these compounds are expected to be hydroxylated at the carbon atom to which X is joined by cytochrome P450, in particular CYP1B1, expressed at high levels in tumors.

The present invention is directed to a new class of prodrugs which are expected to be oxidized in situ by CYP enzymes, in particular enzymes expressed at high levels in tumors. In particular, the prodrugs are believed to be metabolizable by CYP1B1 enzyme. P450 enzymes are involved in Phase I metabolism and are well known to be able to convert an alkene to an epoxide to form an active compound. It is believed that no drugs have previously been activated in this manner. Some of the compounds of the present invention contain nitrogen mustards and may act as alkylating agents.

According to the first aspect of the present invention there is provided novel prodrugs of general formula I or a salt thereof:

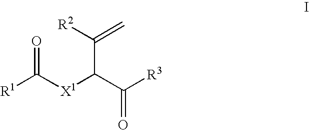

in which X¹ is selected from a group consisting of O, S and NR⁰ in which R⁰ is H or C₁₋₄ alkyl;

R³ is NH₂, NHR⁴, SR⁴, OR⁴, CH₂R⁴ or OH;

R¹ is H, C₁₋₄ alkyl, C₁₋₄ substituted alkyl, C₁₋₄ alkoxy, optionally substituted phenyl, C₇₋₁₂ aralkyl, optionally substituted naphthyl, anthranyl, optionally substituted heteroaryl or a ligand;

R² is H, optionally substituted C₁₋₄ alkyl, C₁₋₄ alkoxy, optionally substituted phenyl, C₇₋₁₂ aralkyl, optionally substituted heteroaryl or a ligand;

R⁴ is C₁₋₄ alkyl, C₁₋₄ substituted alkyl, C₁₋₄ alkoxy, optionally substituted phenyl, C₇₋₁₂ aralkyl, optionally substituted heteroaryl, CₙH₂ₙNR⁵R⁶ or a ligand;

in which at least one of R⁵ and R⁶ is (CH₂)₂A¹ or together with the nitrogen to which they are attached form a ring of formula II

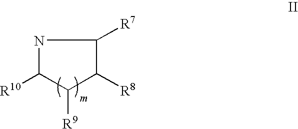

in which at least one of R⁷, R⁸ and R⁹ is selected from A¹ and A¹ substituted C₁₋₄ alkyl, and any others are H or C₁₋₄ alkyl; R¹⁰ is selected from H, C₁₋₄ alkyl, A¹ and A¹ substituted C₁₋₄ alkyl;

A¹ is a leaving group or a halogen atom;

m is 1-4;

n is 1-7;

wherein the substituent groups are selected from C₁₋₄ alkyl, hydroxyl, amino, alkyl amino, halo and aziridine.

Suitable examples of halogen atoms are fluorine, chlorine, bromine and iodine, preferably chlorine. Suitable examples of leaving groups are alkyl or aryl sulphonates, carboxylates, alkyloxy, acyloxy and aryloxy groups.

In the present invention the term ligand includes a group having specific targeting characteristics, useful for instance in antibody or gene-directed enzyme prodrug-type environments. A ligand may be an oligopeptide, biotin, avidin or streptavidin, a polymeric group, an oligonucleotide or a protein. Preferably it has specific binding characteristics and is preferably an antibody or fragment, an antigen, a sense or anti-sense oligonucleotide, or one of avidin, streptavidin and biotin, that is one component of a specific binding pair. Alternatively, it may be a group designed for passive targeting, such as a polymeric group, or a group designed to prolong the stability or reduce immunogenicity such as a hydrophilic group. U.S. Pat. No. 5,843,937 discloses suitable ligands for conjugating to these types of actives and methods for carrying out the conjugation.

In these compounds, the group R¹ is chosen so that it facilitates the intercalation of the compound into DNA. For optimized DNA binding ability, the group R¹ is an aryl group and is preferably selected from the group consisting of optionally substituted phenyl, optionally substituted naphthyl, anthranyl and optionally substituted heteroaryl. When R¹ is optionally substituted naphthyl, excellent intercalation is observed.

A preferred group for R¹ is III

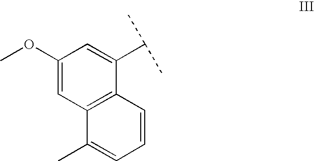

In the compounds of the present invention X¹ is preferably oxygen, although sulphur and nitrogen analogues have been generated and have useful properties.

In one preferred embodiment X¹ is O, R² is CH₃ and R³ is NH₂.

Two examples of such a class of compounds are

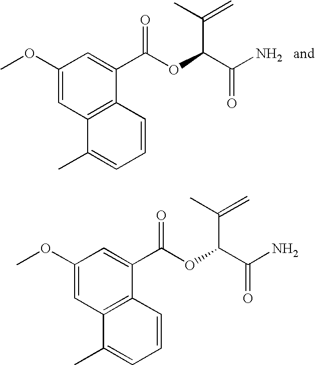

Amide analogues of these compounds have been generated and represent a further embodiment of the present invention. The following allylglycine derivative exemplifies this embodiment:

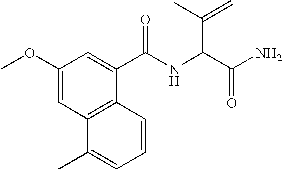

The compounds of the present invention may be present as racemic mixtures or as isolated R or S enantiomers. It is often found that one enantiomer shows more biological activity than another and is therefore preferred.

These compounds are converted into epoxides in vivo by a CYP-mediated biooxidative process. This is shown in the diagram below.

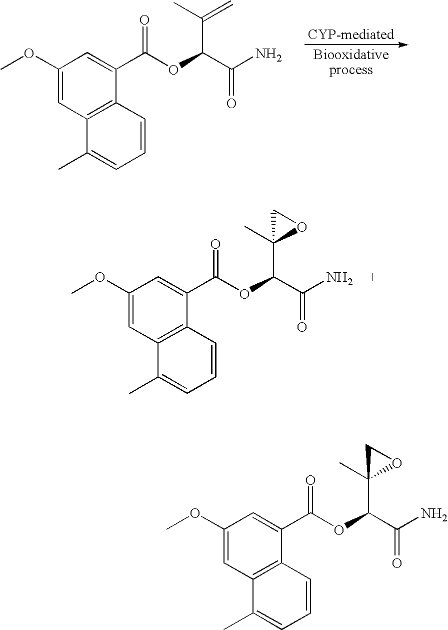

The activated products, the epoxides, of this preferred class of compounds of the invention monoalkylate DNA through the epoxide at the N7 of guanine in the major groove. Nitrogen mustards, that alkylate DNA through the mustard moiety but have the potential to become crosslinking agents via formation of an epoxy group form preferred embodiments of the present invention. Although nitrogen mustards themselves have potent anti-tumour activity, it is believed that conversion to a crosslinking agent through CYP-mediated bioxidation could lead to enhancement of activity or a change in the relative spectrum of activity of a compound.

Accordingly, a second class of preferred compounds of the present invention of general formula I have R³═NHR⁴, wherein R⁴ is a group of formula CₙH₂ₙNR⁵R⁶ as defined above. R⁵ and R⁶ may be joined to form a ring of general formula II. The compounds of the present invention may be pyrrolidine derivatives, that is in which m=1. Another class of compounds of the invention are piperidine derivatives, in which m=2.

In a preferred class of such compounds of the present invention

i) R⁷ is CH₂A¹ and R⁸ is H; or

ii) R⁷ is H and R⁸ is A¹.

In this embodiment R¹⁰ is H or is the same group as R⁷ and the or each R⁹ is H or the same group as R⁸. Such compounds have been shown to cross-link duplex DNA at concentrations similar to those given for the natural products Azinomycin A and B or close analogues. However, the compounds of the present invention are more stable and therapeutically robust, showing greater potential as anti-tumour agents.

Compounds in which the groups R⁷ and R⁸ are not one of the definitions mentioned above in connection with alkylating agents, may nevertheless bind to DNA and cause cytotoxicity.

A preferred structure of group CₙH₂ₙNR⁵R⁶, wherein n=2 is shown below.

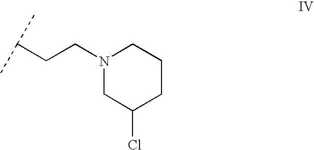

A specific example of this second class of compounds of formula I which contains a nitrogen mustard and may be biooxidatively activated is

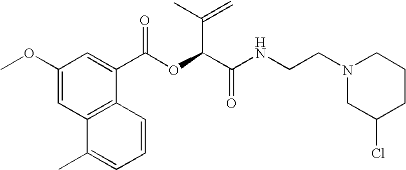

One particular isoform of the cytochrome P450 family of enzymes, CYP1B1, is thought to be tumour specific. This provides for a self-targeting drug delivery system in which a non-toxic (or negligibly cytotoxic) compound can be administered to a patient, for example, in a systematic manner, the compound then being activated at the site of the tumour cells to form a highly cytotoxic compound which acts to kill the tumour cells.

According to the present invention there is also provided a synthetic method in which a compound of formula V

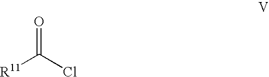

in which R¹¹ is selected from a group consisting of H, C₁₋₄ alkyl, C₁₋₄ substituted alkyl, C₁₋₄ alkoxy, optionally substituted phenyl, C₇₋₁₂ aralkyl, optionally substituted naphthyl, anthranyl and optionally substituted heteroaryl

is reacted with a compound of formula VI

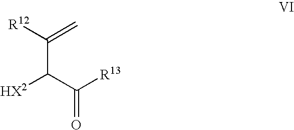

in which R¹² is H, optionally substituted C₁₋₄ alkyl, C₁₋₄ alkoxy, optionally substituted phenyl, C₇₋₁₂ aralkyl, optionally substituted heteroaryl or a ligand;

X² is O, NH or S;

R¹³ is OH, C₁, C₁₋₄ alkoxy or OPG wherein PG is a protecting group;

such that Cl in V is replaced in a nucleophilic substitution reaction by a group of formula VII

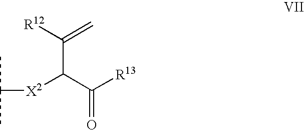

The group R¹³ preferably incorporates a protecting group to ensure that the X² substituent acts as the nucleophilic end of the molecule. Suitable protecting groups for alcohols include benzyl ether, trialkyl silyl (e.g. TBDMS) and tetrahydropyranyl (THP). Of these, benzyl ether is preferred.

Once the coupling is complete the protecting group may be removed by a deprotection reaction. In a preferred embodiment, the protecting group is benzyl ether and this may be removed using H₂ over a Pd/C catalyst or by using HBr reagent to yield a carboxylic acid.

The carboxylic acid may then be reacted with a suitable nucleophile, HR¹⁴, wherein R¹⁴ is selected from the group consisting of NH₂, NHR¹⁵, SR¹⁵ and OR¹⁵ to give a compound of formula VIII

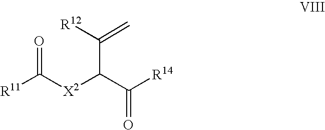

wherein R¹⁵ is selected from the group consisting of C₁₋₄alkyl, C₁₋₄ substituted alkyl, C₁₋₄ alkoxy, optionally substituted phenyl, C₇₋₁₂ aralkyl, optionally substituted heteroaryl, CₚH₂ₚNR¹⁶R¹⁷ and a ligand;

in which at least one of R¹⁶ and R¹⁷ is (CH₂)₂A² or together with the nitrogen to which they are attached form a ring of formula IX

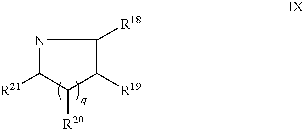

in which at least one of R¹⁸, R¹⁹ and R²⁰ is selected from A² and A² substituted C₁₋₄ alkyl, and any others are H or C₁₋₄ alkyl;

R²¹ is selected from H, C₁₋₄ alkyl, A² and A² substituted alkyl;

A² is a leaving group, hydroxyl, protected hydroxyl or a halogen atom;

q is 1-4;

p is 1-7;

wherein the substituent groups are selected from C₁₋₄ alkyl, hydroxyl, amino, alkyl amino, halo and aziridine.

The product of the above synthetic method may be oxidized at the alkene to which R¹² is attached to form the corresponding active compound. Suitable reagents for carrying out this conversion include Dimethyl dioxirane (DMDO), hydrogen peroxide, the peroxycarboxylic acids and the peroxy-acids, for example meta-chloroperbenzoic acid.

In a synthesis of compounds of the present invention which contain the ring of formula IX, or a CₚH₂ₚNR¹⁶R¹⁷ group, the groups R¹⁶-R²¹ may be the same as in the desired end product of general formula R⁵-R¹⁰. Alternatively, these groups may be precursors for the desired end groups and may be replaced in a subsequent reaction step or steps to generate the desired substituent. Examples of subsequent reaction steps would be halogenating steps carried out on a hydroxyl, or protected hydroxyl after deprotection, group. In such processes a group A² which is hydroxyl or a protected hydroxyl group, is reacted with a halogenating agent, such as a chlorinating agent, optionally after deprotection, to replace the or each A² by a halogen atom. Preferably this halogen atom is chlorine.

In the synthesis, R¹¹ is preferably optionally substituted phenyl, optionally substituted naphthyl, anthranyl or an optionally substituted heteroaryl and is most preferably a group of formula III.

X² is preferably oxygen, R¹² is preferably methyl and R¹⁴ is preferably NH₂ or CₚH₂ₚNR¹⁶R¹⁷, wherein R¹⁶ and R¹⁷, together with the nitrogen to which they are attached form a ring of formula IX.

Intermediates for the synthesis of the compounds of general formula I of the present invention are believed to be new compounds and may be represented by the general formula X

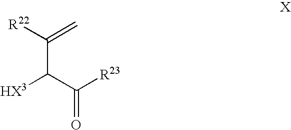

in which X³ is selected from the group consisting of O, NH and S;

R²² is H, C₁₋₄ alkyl, C₁₋₄ alkoxy, optionally substituted phenyl, C₇₋₁₂ aralkyl, optionally substituted heteroaryl or a ligand;

R²³ is C₁₋₄ alkyl, C₁₋₄ substituted alkyl, C₁₋₄ alkoxy, optionally substituted phenyl, C₇₋₁₂ aralkyl, optionally substituted heteroaryl, a ligand or NHR²⁴ wherein R²⁴ is CᵣH₂ᵣNR²⁵R²⁶ or a ligand;

R²⁵ and R²⁶ are (CH₂)₂A³ or both together with the nitrogen to which they are attached, form a ring of formula XI

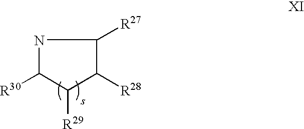

in which at least one of R²⁷, R²⁸ and R²⁹ is selected from A³ and A³ substituted C₁₋₄ alkyl and any others are H or C₁₋₄ alkyl, R³⁰ is selected from H, C₁₋₄ alkyl, A³ and A³ substituted C₁₋₄ alkyl;

A³ is a leaving group, OH, protected hydroxyl or a halogen atom;

s is 1-4;

r is 1-7.

In the intermediates of the present invention, R²² is preferably CH₃. X³ is preferably O and as in the compounds of the present invention of general formula I, R²⁵ and R²⁶ preferably form a ring together with the nitrogen to which they are attached, to give a nitrogen mustard.

The groups R²⁷-R³⁰ may be the same or different to the groups R⁷-R¹⁰ in compound II. If different, the groups R²⁷-R³⁰ may be converted to corresponding R⁷-R¹⁰ in a subsequent reaction step.

A specific example of novel intermediate is

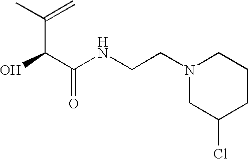

The first aspect of the present invention provides novel prodrugs which preferably have a DNA-intercalating group R¹ and a nitrogen mustard which alkylates DNA. The second aspect of the invention provides a further class of compounds which also have a DNA-intercalating group and a nitrogen mustard. We believe that this second class of compounds is new, even if the compounds do not have an alkene which allows them to act as a prodrug. The oxidised compounds of the first aspect of the invention, the epoxides, fall with the scope of the second aspect of the invention.

According to the second aspect of the present invention there is provided a novel compound of general formula XII or a salt thereof:

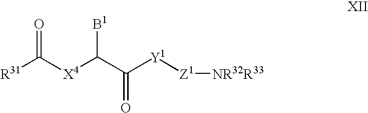

in which X⁴ is selected from the group consisting of O, S, and NR³⁸ in which R³⁸ is H, C₁₋₄alkyl or is linked to B¹;

R³¹ is optionally substituted phenyl, optionally substituted napthyl, anthranyl or optionally substituted heteroaryl;

Y¹ is NH, NR³⁹, S, O or CH₂ wherein R³⁹ is C₁₋₄alkyl;

Z¹ is C₁₋₇ alkanediyl;

B¹ is H, C₁₋₇ alkyl, C₁₋₇ substituted alkyl, C₁₋₇ alkenyl, C₁₋₇ substituted alkenyl, C₁₋₇ alkoxy, optionally substituted phenyl, C₇₋₁₂ aralkyl, optionally substituted heteroaryl, epoxy, optionally substituted epoxy alkyl, aziridine, a ligand, or is a C₁₋₇ optionally substituted alkenylene joined to X⁴ to form a ring;

wherein R³² is (CH₂)₂A⁴ and R³³ is H or the same group as R³², or R³² and R³³ together with the nitrogen to which they are attached for a ring of formula XIII

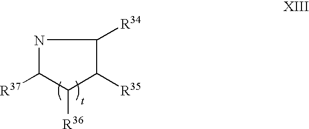

in which R³⁴ is CH₂A⁴ and R³⁵ is H or R³⁴ is H and R³⁵ is A⁴;

R³⁷ is H or the same group as R³⁴ and the or each R³⁶ is H or the same group as R³⁵;

wherein A⁴ is a halogen atom or a leaving group;

t is 1-4;

wherein the substituent groups are selected from C₁₋₄ alkyl, hydroxyl, amino, alkylamino, halo, nitro, cyano, thiol, thiol ether, amide, epoxy, aziridine, carboxylate, carboxylate ester, (CO₂R⁴⁰) sulphoxide (OSO₂R⁴⁰), guinadine, acyl, imidazole, indole, optionally substituted phenyl, alkoxy, aryloxy, acyloxy and acyl amino;

R⁴⁰ is C₁₋₄ alkyl or optionally substituted phenyl.

The group R³¹ is chosen so that it facilitates the intercalation of the compound into DNA. For optimised DNA binding ability, the group R³¹ is an aryl group and may be substituted or include 2 aryl groups joined to one another. When R³¹ is optionally substituted naphthyl, excellent intercalation is observed. A preferred group is III

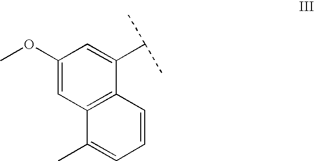

In an embodiment of the present invention B¹ contains an epoxy group of formula XIV

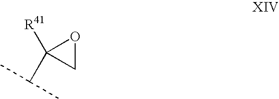

wherein R⁴¹ is selected from the group consisting of H, optionally substituted C₁₋₄ alkyl, C₁₋₄ alkoxy, optionally substituted phenyl, C₇₋₁₂ aralkyl, optionally substituted heteroaryl or a ligand. Preferably R⁴¹ is methyl. Preferably, the epoxy group XIII is a substituent on an alkyl group as B¹, or B¹ is the epoxy group XIV. If the compound, by virtue of its R⁴¹ group, has the ability to intercalate into DNA, the epoxide group is thought to monoalkylate DNA in the major groove at the N7 of guanine, thereby contributing to the compound's anti-tumour activity. However, administering the epoxide may often lead to side effects due to lack of selectivity for cancerous cells.

The present invention also relates to a range of prodrugs which have substantially increased cytotoxicity when activated by oxidation by CYP enzymes. These compounds have an alkene of formula XV in the place of the epoxy group as shown below.

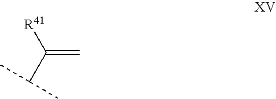

R⁴¹ is selected from the same groups as for XIV above, the corresponding epoxy group.

The alkene is converted to the corresponding epoxide in vivo by a member of the cytochrome P450 family of enzymes. One particular isoform, CYP1B1 is thought to be tumour specific. This provides for self-targeting drug delivery system in which a non cytotoxic (or negligibly cytotoxic) compound can be administered to a patient, for example in a systematic manner, the compound then being activated at the site of the tumour cells to form a highly cytotoxic compound which acts to kill the tumour cells.

The group B¹ may also be selected from the side chains of a naturally occurring amino acid, as shown below:

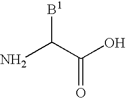

Compounds in which B¹ is not XV do not have the potential for bioxidative activation to form an alkylating group, but still monoalkylate DNA by virtue of the nitrogen mustard (i.e. the group NR³²R³³). The nitrogen mustard replaces the aziridine of the natural product.

In a preferred embodiment of the present invention R³² and R³³, together with the nitrogen to which they are attached form a ring of formula XIII. In this ring, R³⁴ is preferably CH₂A⁴ and R³⁵ is H. In these compounds, the ring is preferably a piperidine derivative (t=2) and R³⁷ is CH₂A⁴, in which A⁴ is the same A⁴ as in R³⁴. Preferably, A⁴ is chlorine. Preferably, in such classes of compounds, B¹ is also an epoxide or alkene as described previously, in order that the compound may act as a DNA cross-linking agent by providing 2 points of attachment for the DNA helix. Such compounds have been shown to crosslink duplex DNA at concentrations similar to those given for the natural products Azinomycin A and B or close analogues. However, the compounds of the present invention are more stable and therapeutically robust, showing greater potential as anti-tumour agents.

Suitable examples of halogen atoms are fluorine, chlorine, bromine and iodine, preferably chlorine. Suitable examples of leaving groups as A⁴ are carboxylates, alkyl sulphonates, aryl sulphonates, alkyloxy, acyloxy and aryloxy groups.

In the compounds of the present invention X⁴ is preferably oxygen, although sulphur and nitrogen analogues have been generated and have useful properties.

The compounds of the present invention may be pyrrolidine derivatives, that is in which t=1. Another class of compounds of the invention are piperidine derivatives, in which t=2.

In preferred compounds of the present invention Y¹ is NH and Z¹ is (CH₂)₂. The following compound has shown excellent anti-tumour activity in a NCl60 cell line.

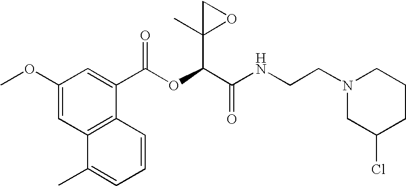

The compounds of the present invention may be present as racemic mixtures or as isolated R- or S-enantiomers. It is often found that one enantiomer shows more biological activity and is therefore preferred.

The methods for synthesising the compounds XII are generally conventional. Preferably the compounds are made by producing a precursor cyclic amino alkylamine and reacting this in a nucleophilic substitution reaction with an appropriately activated carboxylic acid or derivative. The OH of the carboxylic acid may be made into a good leaving group for the reaction by adding acid to the reaction or alternatively by converting the acid into an acyl chloride.

According to the present invention there is provided a synthetic method in which a compound of formula XVI

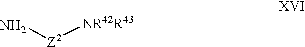

wherein Z² is C₁₋₇ alkanediyl;

R⁴² is (CH₂)₂A⁵ and R⁴³ is H or the same group as R⁴², or R⁴² and R⁴³ together with the nitrogen to which they are attached form a ring of formula XVII

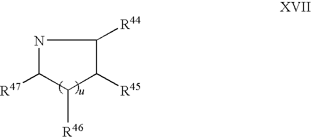

in which R⁴⁴ is CH₂A⁵ and R⁴⁵ is H or R⁴⁴ is H and R⁴⁵ is A⁵;
R⁴⁷ is H or the same group as R⁴⁴ and the or each R⁴⁶ is H or the same group as R⁴⁵;
u is 1-4;
A⁵ is a leaving group, hydroxyl, protected hydroxyl or a halogen atom; is reacted with a compound of formula XVIII

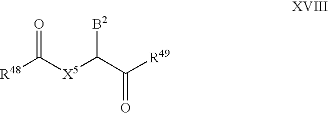

such that R⁴⁹ is replaced by XIX

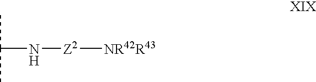

wherein R⁴⁹ is selected from the group consisting of a leaving group or a halogen;

in which X⁵ is selected from the group consisting of O, S and NR⁴⁶ in which R⁴⁶ is H or C₁₋₄ alkyl or is linked to B²;

R⁴⁸ is optionally substituted phenyl, optionally substituted naphthyl, anthranyl or optionally substituted heteroaryl;

B² is selected from the same group as B¹ with the proviso that the substitutent groups may be protected.

In this method, the groups R⁴²-R⁴⁷ may be the same as in the desired end product of the general formula R³²-R³⁷. Alternatively, these groups may be precursors for the desired end groups and may be reacted in a subsequent reaction step or steps to generate the desired substituent R³² to R³⁷. Examples of subsequent reaction steps would be halogenating steps, carried out on a hydroxyl, or protected hydroxyl after deprotection, group. In such processes a group A⁵ which is a hydroxyl or a protected hydroxyl group, is reacted with a halogenating agent, such as a chlorinating agent, optionally after deprotection to replace the or each A⁵ group by a halogen atom. Preferably this halogen atom is chlorine.

In the method, the cyclic amino alkyl amines are commercially available or may be synthesized in preliminary steps.

Suitable protecting groups for alcohols include benzyl ether, trialkyl silyl (e.g. TBDMS) and tetrahydropyranyl (THP). Of these, benzyl ether is preferred.

In the synthesis, B² is preferably an epoxide of general formula XIV or an alkene of general formula XV. Preferably R⁴⁸ is optionally substituted naphthyl, more particularly a group of general formula III.

In further preferred embodiments of the method of the present invention, X⁵ is O, Y¹ is NH and Z¹ is (CH₂)₂.

The compounds of the present invention of general formula I and XII may be useful in a method of treatment of an animal by therapy. In particular, the cytotoxic properties of the compound itself or the activated form, as the case may be, may be useful in anti-tumour treatment. The invention further provides the use of these compounds in the manufacture of compositions for use in a method of treatment of an animal. The compounds may be incorporated into a pharmaceutical composition together with a pharmaceutically acceptable excipient.

Pharmaceutical compositions may be suitable for intramuscular, intraperitoneal, intrapulmonary, oral or, most preferably, intravenous administration. The compositions may contain suitable matrixes, for example for controlled or delayed release. The compositions may be in the form of solutions, solids, for instance powders, tablets or implants, and may comprise the compound of the formula I in solid or dissolved form. The compound may be incorporated in a particulate drug delivery system, for instance in a liquid formulation. Specific examples of suitable excipients include lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including araboc and tragacanth; and proteins, such as gelatin and collagen. If desired, disintegrating or solubilising agents may be added, such as the cross-linked polyvinyl pyrrolidone, agar, and alginic acid or a salt thereof, such as sodium alginate. Solid compositions may take the form of powders and gels but are more conveniently of a formed type, for example as tablets, cachets or capsules (including spansules). Alternative, more specialised types of formulation include liposomes, nanosomes and nanoparticles.

The animal which is treated is generally human, although the compounds may also have veterinary use. The indication treated is generally cancer, including adenocarcinoma, leukemia, lymphoma, melanoma, myeloma, sarcoma, teracarinoma, and, in particular, cancers of the adrenal gland, bladder, bone, bone marrow, brain, breast, cervix, gall bladder, ganglia, gastrointestinal tract, heart, liver, kidney, lung, muscle, ovary, pancreas, parathyroid, penis, prostate, salivary glands, skin, spleen, testes, thymus, thyroid and uterus. The tumour may, for instance, be defined as a tumour expressing high levels of CYP1B1.

The oxidised forms of the prodrugs of the first aspect of the present invention and the mustard compounds of the second aspect of the invention alkylate DNA and cause cytotoxicity. As such, they are potent cytotoxic agents whose exact biological mechanism of action is unknown but involves the disruption of template and other functions of DNA. General inhibition of template function of DNA will affect all dividing cells in the body and lead to unacceptable side effects in a therapeutic setting. However, the targeted production of the epoxide forms only in tumour cells that over express particular isoforms of cytochrome P450 will lead to a specific cytotoxic effect only in those cells.

### BRIEF DESCRIPTION OF THE FIGURES

FIG. 1: Effect of 2 on the electrophoretic mobility of F174 plasmid DNA.

FIG. 2: (a) cytotoxicity of 2 on CHO cells (with or without CYP3A4).
(b) cytotoxicity of mach 361/1 ((25,35)-(1)) on CHO cells (with or without CYP3A4).

FIG. 3: (a): Effect of 20 on DNA crosslinking after 1 h incubation with pUC18 plasmid DNA.
(b) The percentage crosslinked (double stranded) DNA.

FIG. 4: As for FIG. 3 but after 2 h incubation.

FIG. 5: As for FIG. 3 but after 3 h incubation.

The following examples illustrate the invention:

### Example 1

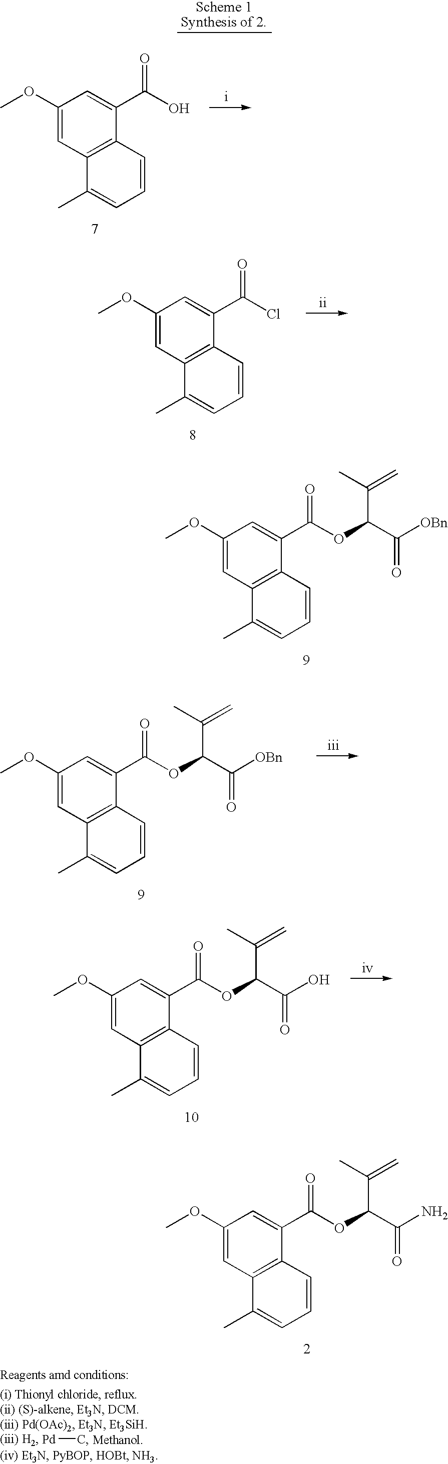

The novel alkene amides of general formula I were prepared from the carboxylic acid 7 in four steps. The acid chloride 8 was coupled to the benzyl hydroxybutenoate by dropwise addition to a stirred solution of alcohol together with Et₃N in dry CH₂Cl₂ under a nitrogen atmosphere at 0° C. After 4 h the reaction was quenched with H₂O, extracted with CH₂Cl₂ and purified to give 9 in 65% yield. Proton NMR analysis confirmed the structure and showed the alkenyl protons as multiplets at 5.33 and 5.18 ppm. The benzyl CH₂ protons also appeared as a multiplet at 5.31 ppm, the H-2 proton was detected at 5.77 ppm and the methyl hydrogens had values of 2.68 ppm for the aromatic methyl and 3.96 ppm for the methoxy methyl.

The benzyl group was selectively deprotected using catalytic Pd(OAc)₂. A solution of the Pd(OAc)₂, Et₃N and Et₃SiH in dry CH₂Cl₂ was stirred at RT under N₂ for 15 min. A solution of the ester 9 in dry CH₂Cl₂ was then added dropwise. The mixture was stirred at RT overnight before quenching the reaction by the addition of NH₄Cl. After extraction with Et₂O the alkenyl carboxylic acid was recovered in 90% yield. This acid was then treated with 35% NH₃, Et₃N, HOBt and PyBOP to give the amide 2 in 66% yield. NMR analysis showed the NH₂ protons as broad singlets at 6.13 and 5.65 ppm whereas the H-2 proton appeared at 5.87 ppm. The alkene methylene protons were identified as two multiplets at 5.36 and 5.21 ppm, and the methyl groups as singlets at 3.95 (OCH₃), 2.52 (Ar—CH₃) and 1.96 ppm (CH₃). The aromatic protons on the naphthalene chromophore were at 8.65 (1H), 7.90 (1H), 7.50 (1H) and 7.36 ppm (2H). The stereoisomer, compound (R)-2 was synthesised using the same route but employing (R)-hydroxy butenoate.

### Example 2

### Preliminary Biological Investigations of Potential Bio-Oxidative Prodrugs

Initial cytotoxicity studies were in the performed U2-OS and HoeR cell lines. The U2-OS is a human osteosarcoma cell line and HoeR is a DNA minor groove binder-resistant variant of this. Both are available from the American Type Culture Collection (ATCC), Dr. Raymon H. 10801 University Boulevard, Manassas, Va., 20110-2209, USA. The studies revealed that the alkene amide analogues 2 and (R)-2 were not cytotoxic compounds whereas their epoxide counterparts (2S, 3S)-1, (2S, 3R)-1, (2R, 3R)-1, (2R, 3S)-1 demonstrated good activity in these cell lines (Table 1).

TABLE 1
IC₅₀ values in U2-OS and HoeR. U2 OS is a human osteosarcoma cell
line, HoeR is a Hoechst415 resistant version of U2-OS.

| | |
|---|---|
| | 1 |
| 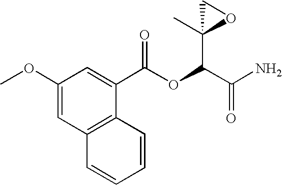 | |

| | IC₅₀ (nM) of compound | | | | | |
|---|---|---|---|---|---|---|
| Panel/ | (2S, | (2S, | (2R, | (2R, | | |
| Cell line | 3S)-1 | 3R)-1 | 3R)-1 | 3S)-1 | (2S)-2 | (2R)-2 |
| U2-OS | 15 | 120 | 40 | 40 | >10 000 | >10 000 |
| HoeR | 14 | 121 | 40 | 45 | >10 000 | >10 000 |

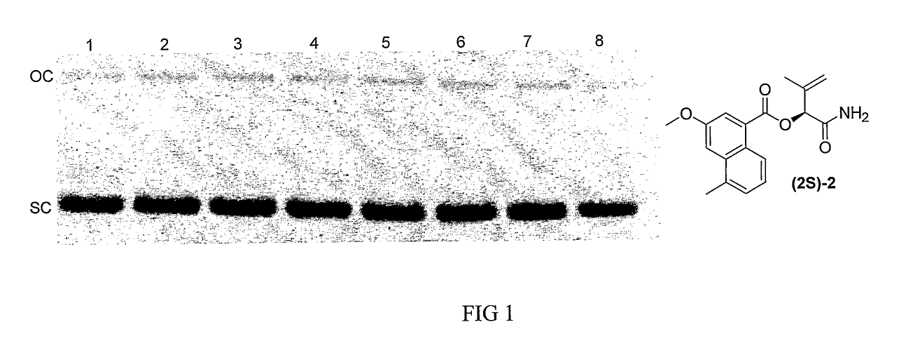

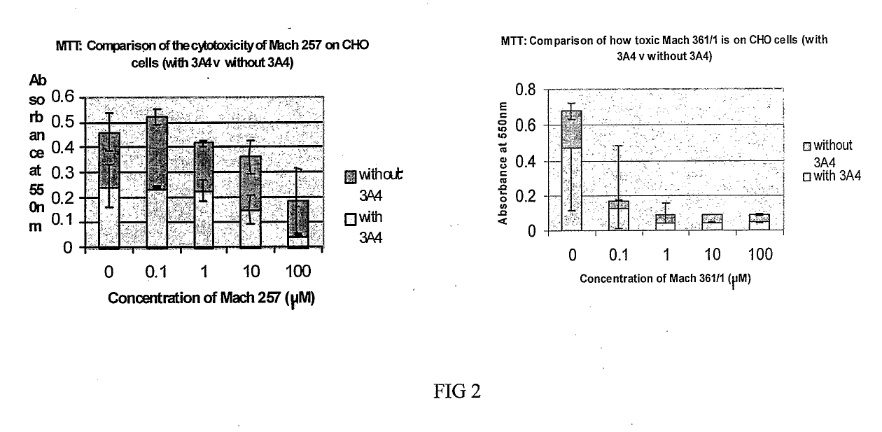

FIG. 1 shows the effect of 2 on the electrophoretic mobility of F174 plasmid DNA. Lane 1 contains DNA only, lanes 2-8 have 10⁻³, 10⁻², 10⁻¹, 1, 10, 20 and 30 drug/bp ratio respectively. The DNA concentration was 3.8 μm and SC stands for supercoiled DNA and OC for Open Circular DNA.

### Example 3

### Preliminary Metabolism Studies

Table 1 shows that 2 lacks cytotoxic activity in U2-OS and HoeR cell lines in vitro at concentrations as high as 10 μM. Further studies of 2 in wild type CHO cells and CHO cells that have been transfected with CYP3A4 revealed that the prodrug 2 appears more cytotoxic in CYP3A4 CHO cells compared to wild type (absent in CYP3A4) FIG. 2(a) shows the cytotoxicity of 2 on CHO cells, with or without CYP3A4.

FIG. 2(b) similarly shows the cytotoxicity of (25, 35)-1. By comparison the epoxide (active) compound has high cytotoxicity in either cell line. This is an initial indicator that shows that the alkene functionality can indeed be metabolised by cytochrome P-450 enzymes to a compound, which is more cytotoxic than the parent alkene precursor.

### Example 4

The synthesis of a compound of general formula XII was carried out according to schemes 2 and 3.

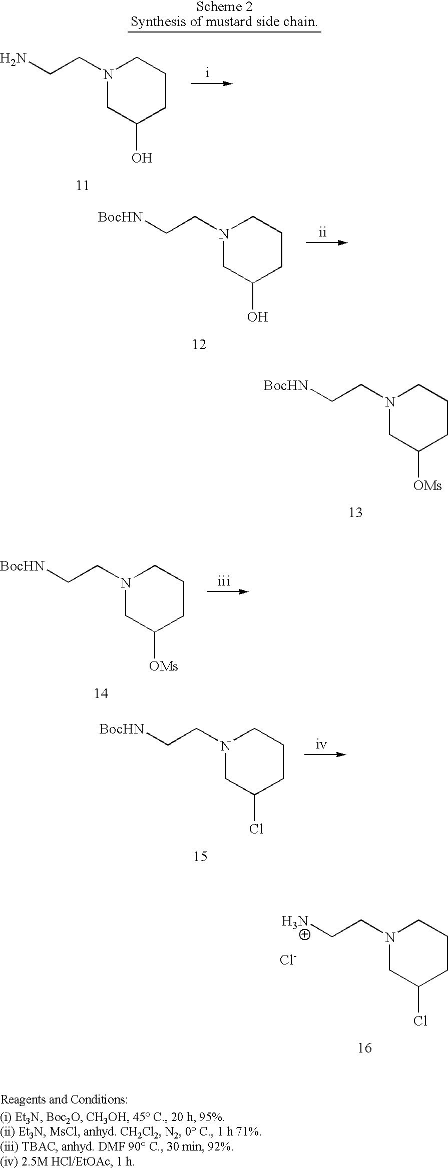

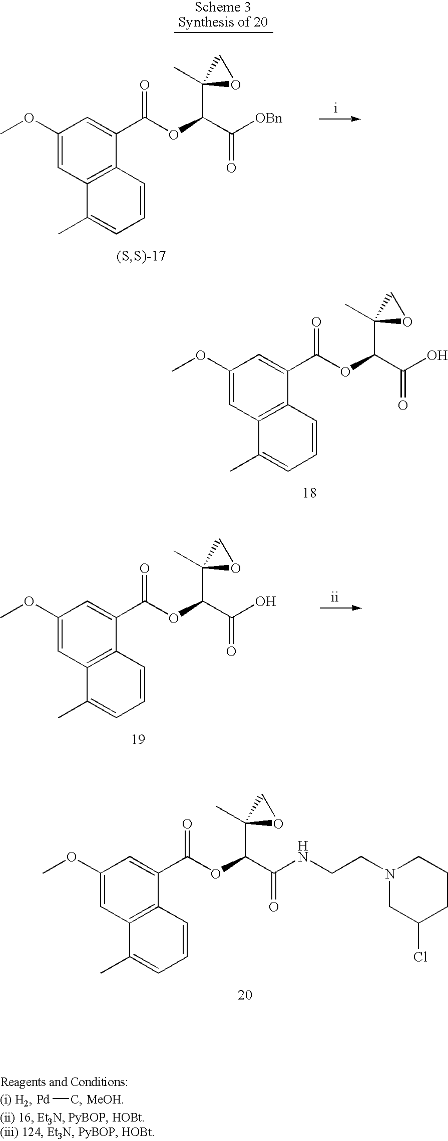

The 2-chloropiperidine 16 was synthesised from 1-(2-aminoethyl)-piperidin-3-ol 11 following Boc-protection of the primary amine. This was achieved by stirring the diamino alcohol 11 in CH₃OH for 5 min after which Boc₂O (dissolved in CH₃OH) was added dropwise over 20 min and the reaction mixture stirred at 45° C. for 20 h. It was concentrated in vacuo, dissolved in EtOAc and washed with H₂O to afford 12 as a straw coloured oil in 95% yield.

The Boc-protected amine 12 was then converted to the mesylate 13 by stirring in anhyd. CH₂Cl₂, with Et₃N and adding MsCl dropwise at 0° C. After 1 h the reaction was quenched with ice cold NaHCO₃ in brine and extracted with cold CH₂CO₂ to give 14 the precursor to the Boc protected 2-chloropiperidine derivative in 71% yield. The mesylate was immediately transformed into the Boc-protected mustard 15 by heating in anhyd. DMF to 90° C. in the presence of TBAC for 30 min after which the DMF was removed in vacuo and the reaction residue redissolved in CH₂Cl₂ and washed with cold NaHCO₃ to give the Boc-protected mustard 15 in 92% yield. Prior to coupling to the carboxylic acid functionality of the left hand portion of the azinomycins, the Boc-protected amine was deprotected by stirring in dry 2.5 M HCl in EtOAc for an hour. EtOAc was then removed by evaporation to give the chloride salt of the amine.

The benzylester (S,S)-17 was synthesised using a stereoselective method as described in Bryant et al in Synlett. 1996, 10, 973. 17 is converted to the free epoxy carboxylic acid in Scheme 3, step (i), by hydrogenolysis using Pd—C in CH₃OH under hydrogen atmosphere.

To prepare 20, the freshly prepared epoxy carboxylic acid was dissolved in dry DMF, stirred at 0° C. and was successively treated with 16, Et₃N and PyBOP. The reaction mixture was then warmed to RT and stirred for 18 h after which toluene was added and the resulting solution successively washed with NaHCO₃ and brine. Column chromatography (10-20% CH₃OH/CH₂Cl₂) provided 20 in 67% yield.

### Example 5

### DNA Crosslinking

Plasmid DNA pUC 18 was linearised by digestion with Hind III. The linear DNA was then dephosphorylated with BAP and ³²P-radiolabelled on the 5′-end. The DNA was then purified by EtOH precipitation to remove unincorporated γ-³²P ATP and the DNA resuspended in sterile double distilled H₂O. To each reaction sample was added ³²P-end labelled DNA and drug at the appropriate concentration. Following incubation at 37° C. for the required time, the reaction was terminated by the addition of Stop Solution Buffer. The DNA-drug adduct was EtOH precipitated and dried by lyophilisation. Each dried DNA sample including an untreated DNA single strand as a control was denatured by resuspending in alkali denaturing buffer. The double stranded control DNA was then dissolved in sucrose loading buffer and the samples loaded and electrophoresed on a 20 cm long 0.8% horizontal agarose gel submerged in 1×TAE buffer at 40 V for 16 h. Gels were then dried and autoradiographed

Compound 20 which consists of both the epoxide and mustard functionality was tested at concentrations between 0.1 and 50 μM at 1 h, 2 h and 3 h intervals. FIG. 3 displays an autoradiograph and a concentration-response curve showing that 20 can imitate the natural product Azinomycin A and crosslinks linear double stranded plasmid pUC18 DNA after one hour incubation. Crosslink formation starts at concentrations as low as 0.1 μM and reaches 100% crosslinking at −10 μM. After incubation for an hour the CR₅₀ (concentration at which 50% of the duplex is crosslinked) was determined to be 3.1 μM. The percentage of crosslinked DNA was determined from autoradiograph densitometry.

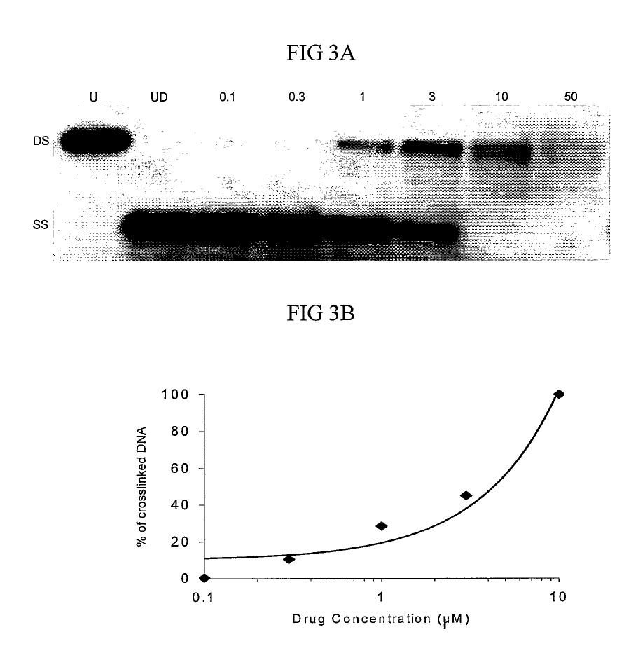

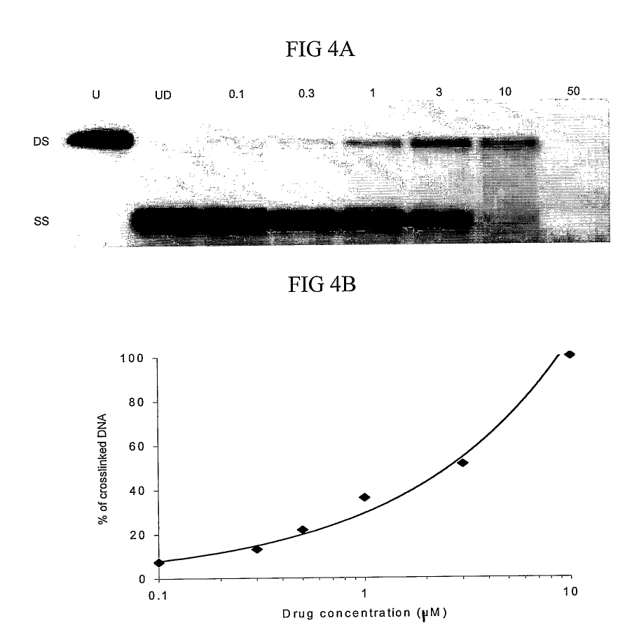

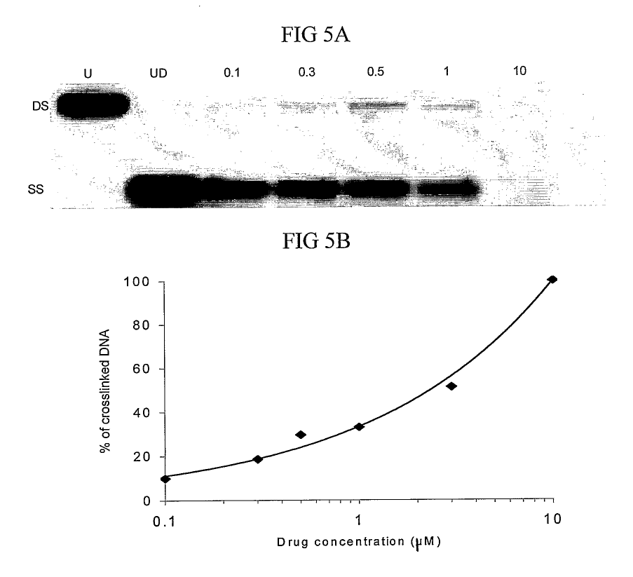

Crosslink formation progressed steadily over time and after 2 h the CR₅₀ was reduced from 3.1 μM to 2.7 μM (FIG. 4). After 3 h the CR₅₀ was 2.2 μM (FIG. 5).

In FIGS. 3-5 DS stands for double stranded DNA, SS stands for single stranded DNA, U for untreated nondenatured DNA and UD for untreated denatured DNA.

### Example 6

### Antitumour Activity

Examples 6 and 7 make use of the NCl 60 cell panel. This is an in vitro cell line screening project providing direct support to the National Cancer Institute's USA Developmental Therapeutic Programme for anti cancer discovery. The methodolgy for the cell line's operation is described by Boyd et al in Drug Development Research 1995, 34, 91-109.

The antitumour activity in the NCl 60 cell line panel shows the compound to have low micromolar to high nanomolar activity. Table 2 shows the anti-tumour activity (GI₅₀, μm) of compound 20, where the GI₅₀ value is the concentration which results in growth inhibition of 50%.

TABLE 2

| | Compound |
|---|---|
| Cell line | 20 |

Leukemia

| | |
|---|---|
| CCRF-CEM | 3.26 |
| HL-60(TB) | 6.98 |
| K562 | 8.33 |
| MOLT-4 | 3 |
| RPMI-8226 | 4.18 |
| SR | 2.02 |

NSCLC

| | |
|---|---|
| A549/ATCC | 31.1 |
| EKVX | 17.5 |
| HOP-62 | 100 |
| HOP-92 | 13.8 |
| NCI-H226 | |
| NCI-H23 | |
| NCI-H322M | 17.9 |
| NCI-H460 | 13 |
| NCI-H522 | 7.77 |

COLON

| | |
|---|---|
| COLO 205 | 13.8 |
| HCC2998 | 8.25 |
| HCT-116 | 100 |
| HCT-15 | |
| HT29 | 11 |
| KM12 | 14.5 |
| SW-620 | 5.26 |

CNS

| | |
|---|---|
| SF-268 | 16.6 |
| SF-295 | 13.1 |
| SF-539 | 6.27 |
| SNB-19 | 14.3 |
| SNB-75 | |
| U251 | 7.82 |

MELAN

| | |
|---|---|
| LOX IMVI | 3.62 |
| MALME-3M | 14.8 |
| M14 | 100 |
| SK-MEL-2 | 18.6 |
| SK-MEL-28 | 11.6 |
| SK-MEL-5 | 1.56 |
| UACC-257 | |
| UCC-62 | 14.7 |

OVAR

| | |
|---|---|
| IGROV1 | 12.1 |
| OVCAR-3 | 18.6 |
| OVCAR-4 | 21.5 |
| OVCAR-5 | 16.1 |
| OVCAR-8 | 44.5 |
| SKOV-3 | 19.7 |

RENAL

| | |
|---|---|
| 786-0 | 100 |
| A498 | 17.7 |
| ACHN | 1.17 |
| CAKI-1 | 10.3 |
| RXF 393 | 17.5 |
| SN12C | 6.76 |
| TK10 | 23.4 |

PROST

| | |
|---|---|
| PC-3 | 11.2 |
| DU-145 | 4.49 |

BREAST

| | |
|---|---|
| MCF7 | 14.7 |
| NCI/ADRRES | 11.1 |
| MDA-MB- | |
| 231/ATCC | 14 |
| HS 578T | 17.5 |
| 435 | 14.3 |
| BT-549 | 12.5 |
| T-47D | 23 |
| MGMID | 12.3 |

### Example 7

A similar method to that used in Example 4 (Schemes 2 and 3) was used to synthesise the alkylating analogue using the mustard 16 and alkene carboxylic acid 10 to give 21 (59% yield).

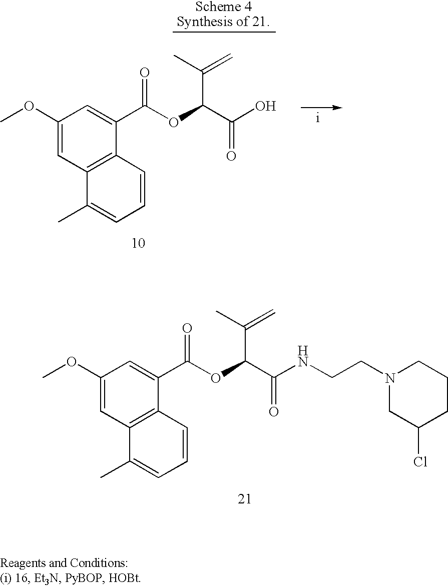

### Antitumour Activity

Table 3 shows the anti-tumour activity (GI₅₀, μM) of 21.

TABLE 3

| | Compound |
|---|---|
| Cell line | 21 |

Leukemia

| | |
|---|---|
| CCRF-CEM | 3.24 |
| HL-60(TB) | 12.1 |
| K562 | 5.16 |
| MOLT-4 | 3.3 |
| RPMI-8226 | 3.43 |
| SR | 2.34 |

NSCLC

| | |
|---|---|
| A549/ATCC | 8.26 |
| EKVX | 11.4 |
| HOP-62 | 3.45 |
| HOP-92 | 4.12 |
| NCI-H226 | |
| NCI-H23 | |
| NCI-H322M | 8.07 |
| NCI-H460 | 6.26 |
| NCI-H522 | 5.1 |

COLON

| | |
|---|---|
| COLO 205 | 6.08 |
| HCC2998 | 4.27 |
| HCT-116 | 12.8 |
| HCT-15 | 0.049 |
| HT29 | 7.26 |
| KM12 | 9.3 |
| SW-620 | 2.76 |

CNS

| | |
|---|---|
| SF-268 | 11.9 |
| SF-295 | 4.35 |
| SF-539 | 0.45 |
| SNB-19 | 5.63 |
| SNB-75 | 1.2 |
| U251 | 2.63 |

MELAN

| | |
|---|---|
| LOX IMVI | 2.84 |
| MALME-3M | 7.51 |
| M14 | 1.75 |
| SK-MEL-2 | 7.81 |
| SK-MEL-28 | 8.48 |
| SK-MEL-5 | 1.99 |
| UACC-257 | |
| UCC-62 | 4.41 |

OVAR

| | |
|---|---|
| IGROV1 | 4.21 |
| OVCAR-3 | 10.2 |
| OVCAR-4 | 20.9 |
| OVCAR-5 | 11.1 |
| OVCAR-8 | 32.9 |
| SKOV-3 | 17.2 |

RENAL

| | |
|---|---|
| 786-0 | 4.48 |
| A498 | 0.19 |
| ACHN | 1.78 |
| CAKI-1 | 5.66 |
| RXF 393 | 19.1 |
| SN12C | 3.76 |
| TK10 | 12.1 |

PROST

| | |
|---|---|
| PC-3 | 7.96 |
| DU-145 | 5.15 |

BREAST

| | |
|---|---|
| MCF7 | 4.62 |
| NCI/ADRRES | 12.8 |
| MDA-MB- | |
| 231/ATCC | 11 |
| HS 578T | 3.69 |
| 435 | 7.62 |
| BT-549 | 4.55 |
| T-47D | 10.8 |
| MGMID | 5.12 |

The results from examples 6 and 7 show that compounds 20 and 21 have significant cytotoxicity in the low micromolar range across a wide range of human tumour cell lines.

### Example 8

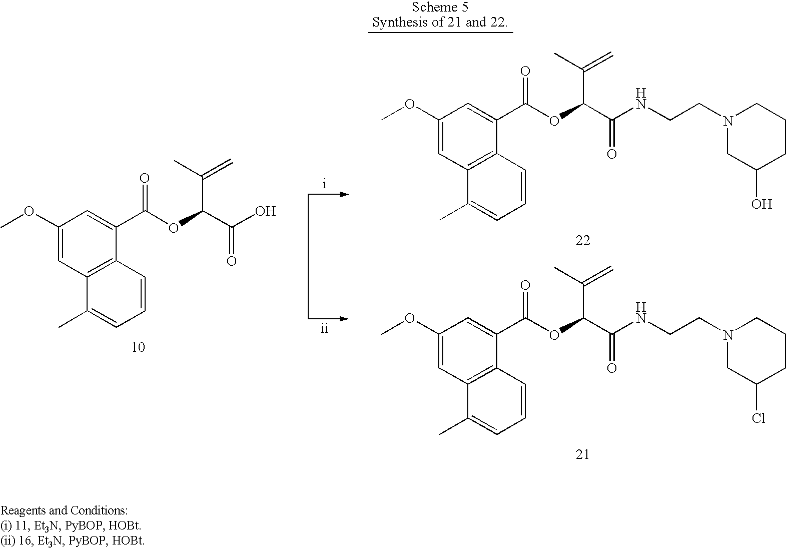

To synthesise the non-alkylating analogue 22, compound 10 the carboxylic acid intermediate was coupled with hydroxypiperidine 11 in 66% yield using PyBOP methodology. 21 was synthesised as described in Example 7.

1. A compound of formula I or a salt thereof 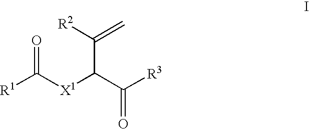
in which X¹ is selected from a group consisting of O, S and NR⁰ in which R⁰ is H or C₁₋₄ alkyl;
R³ is NH₂, NHR⁴, SR⁴, OR⁴, CH₂R⁴ or OH;
R¹ is H, C₁₋₄ alkyl, C₁₋₄ substituted alkyl, C₁₋₄ alkoxy, optionally substituted phenyl, C₇₋₁₂ aralkyl, optionally substituted naphthyl, anthranyl, optionally substituted heteroaryl or a ligand;
R² is H, optionally substituted C₁₋₄ alkyl, C₁₋₄ alkoxy, optionally substituted phenyl, C₇₋₁₂ aralkyl, optionally substituted heteroaryl or a ligand;
R⁴ is C₁₋₄ alkyl, C₁₋₄ substituted alkyl, C₁₋₄ alkoxy, optionally substituted phenyl, C₇₋₁₂ aralkyl, optionally substituted heteroaryl, CₙH₂ₙNR⁵R⁶ or a ligand;
in which at least one of R⁵ and R⁶ is (CH₂)₂A¹ or together with the nitrogen to which they are attached form a ring of formula II 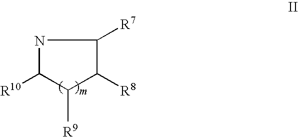
in which at least one of R⁷, R⁸ and R⁹ is selected from A¹ and A¹ substituted C₁₋₄ alkyl, and any others are H or C₁₋₄ alkyl; R¹⁰ is selected from H, C₁₋₄ alkyl, A¹ and A¹ substituted C₁₋₄ alkyl;
A¹ is a leaving group or a halogen atom;
m is 1-4;
n is 1-7;
wherein the substituent groups are selected from C₁₋₄ alkyl, hydroxyl, amino, alkyl amino, halo and aziridine.

2. A compound according to claim 1 in which R¹ is optionally substituted phenyl, optionally substituted naphthyl, anthranyl or optionally substituted heteroaryl.

3. A compound according to claim 1 in which R¹ is III

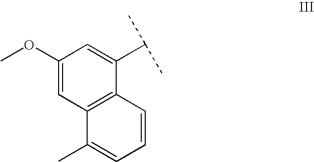

4. A compound according to claim 1 in which X¹ is O.

5. A compound according to claim 1 in which R² is CH₃.

6. A compound according to claim 1 in which R³ is NHR⁴.

7. A compound according to claim 6 in which R⁴ is CₙH₂ₙNR⁵R⁶.

8. A compound according to claim 7 in which CₙH₂ₙNR⁵R⁶ is IV

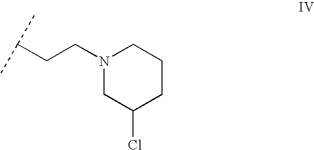

9. A compound according to claim 1 which is

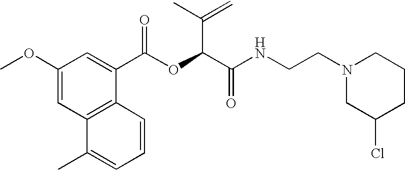

10. A compound according to claim 1 in which R³ is NH₂.

11. A compound according to claim 10 selected from

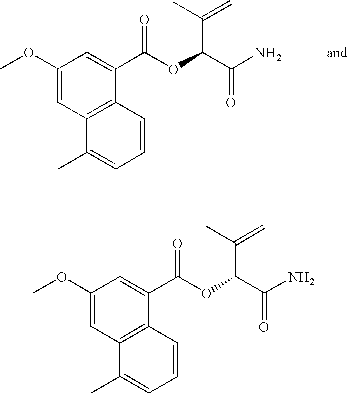

12. A compound according to claim 1 for use in a method of medical treatment of an animal by therapy.

13. Use of a compound according to claim 1 in the manufacture of a composition for use in a method of medical treatment of an animal by therapy, preferably in an anti-tumour treatment.

14. A pharmaceutical composition comprising the compound of claim 1 and a pharmaceutically acceptable excipient.

15. A synthetic method in which a compound of formula V 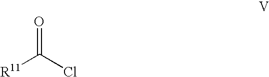
in which R¹¹ is selected from a group consisting of H, C₁₋₄ alkyl, C₁₋₄ substituted alkyl, C₁₋₄ alkoxy, optionally substituted phenyl, C₇₋₁₂ aralkyl, optionally substituted naphthyl, anthranyl and optionally substituted heteroaryl
is reacted with a compound of formula VI 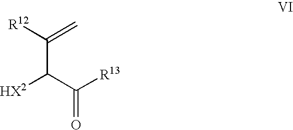
in which R¹² is H, optionally substituted C₁₋₄ alkyl, C₁₋₄ alkoxy, optionally substituted phenyl, C₇₋₁₂ aralkyl, optionally substituted heteroaryl or a ligand;
X² is O, NH or S;
R¹³ is OH, Cl, C₁₋₄ alkoxy or OPG wherein PG is a protecting group;
such that CI in V is replaced in a nucleophilic substitution reaction by a group of formula VII

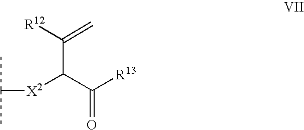

16. A method according to claim 15 followed by removal of the protecting group to give R¹³═OH.

17. A method according to claim 16 followed by reaction with a group of formula HR¹⁴ to give a compound of formula VIII 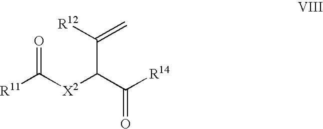
wherein R¹⁴ is selected from the group consisting of NH₂, NHR¹⁵, SR¹⁵ and OR¹⁵;
R¹⁵ is C₁₋₄ alkyl, C₁₋₄ substituted alkyl, C₁₋₄ alkoxy, optionally substituted phenyl, C₇₋₁₂ aralkyl, optionally substituted heteroraryl, CₚH₂ₚNR¹⁶R¹⁷ or a ligand;
in which at least one of R⁶ and R¹⁷ is (CH₂)₂A² or together with the nitrogen to which they are attached form a ring of formula IX 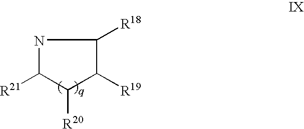
in which at least one of R¹⁸, R¹⁹ and R²⁰ is selected from A² and A² substituted C₁₋₄ alkyl, and any others are H or C₁₋₄ alkyl;
R²¹ is selected from H, C₁₋₄ alkyl, A² and A² substituted alkyl;
A² is a leaving group, halogen atom, hydroxyl or a protected hydroxyl;
q is 1=4;
p is 1-7;
wherein the substituent groups are selected from C₁₋₄ alkyl, hydroxyl, amino, alkyl amino, halo and aziridine.

18. A method according to claim 15 wherein the protecting group is benzyl.

19. A method according to claim 15 in which X² is O.

20. A method according to claim 15 in which R¹² is CH₃.

21. A method according to claim 15 in which R¹¹ is III

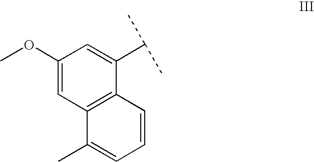

22. A method according to claim 15 in which the alkene to which R¹² is attached is oxidized to the corresponding epoxide.

23. A compound of general formula X 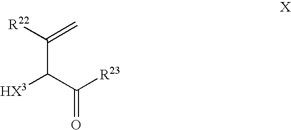
in which X³ is selected from the group consisting of O, NH and S;
R²² is H, C₁₋₄ alkyl, C₁₋₄ alkoxy, optionally substituted phenyl, C₇₋₁₂ aralkyl, optionally substituted heteroaryl or a ligand;
R²³ is C₁₋₄ alkyl, C₁₋₄ substituted alkyl, C₁₋₄ alkoxy, optionally substituted phenyl, C₇₋₁₂ aralkyl, optionally substituted heterorayl, a ligand or NHR²⁴ wherein R²⁴ is CᵣH₂ᵣNR²⁵R²⁶ or a ligand;
R²⁵ and R²⁶ are (CH₂)₂A³ or together with the nitrogen to which they are attached form a ring of formula XI 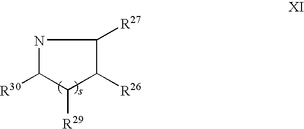
in which at least one of R²⁷, R²⁸ and R²⁹ is selected from A³ and A³ substituted C₁₋₄ alkyl and any others are H or C₁₋₄ alkyl, R³⁰ is selected from H, C₁₋₄ alkyl, A³ and A³ substituted C₁₋₄ alkyl;
A³ is a leaving group, halogen atom, hydroxyl or protected hydroxyl;
s is 1-4;
r is 1-7.

24. A compound of general formula XII or a salt thereof 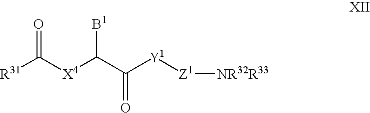
in which X⁴ is selected from the group consisting of O, S, and NR³⁸ in which R³⁸ is H, C₁₋₄ alkyl or is linked to B¹;
R³¹ is optionally substituted phenyl, optionally substituted napthyl, anthranyl or optionally substituted heteroaryl;
Y¹ is NH, NR³⁹S, O or CH₂ wherein R³⁹ is C₁₋₄ alkyl;
Z¹ is C₁₋₇ alkanediyl;
B¹ is H, C₁₋₇ alkyl, C₁₋₇ substituted alkyl, C₁₋₇ alkenyl, C₁₋₇ substituted alkenyl, C₁₋₇ alkoxy, optionally substituted phenyl, C₇₋₁₂ aralkyl, optionally substituted heteroaryl, epoxy, optionally substituted epoxy alkyl, aziridine, a ligand, or is a C₁₋₇ optionally substituted alkenylene joined to X⁴ to form a ring;
wherein R³² is (CH₂)₂A⁴ and R³³ is H or the same group as R³², or R³² and R³³ together with the nitrogen to which they are attached for a ring of formula XIII 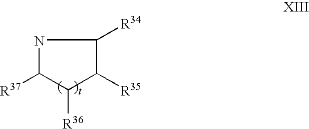
in which R³⁴ is CH₂A⁴ and R³¹ is H or R³⁴ is H and R³⁵ is A⁴;
R³⁷ is H or the same group as R³⁴ and the or each R³⁶ is H or the same group as R³⁵,
wherein A⁴ is a halogen atom or a leaving group;
t is 1-4;
wherein the substituent groups are selected from C₁₋₄ alkyl, hydroxyl, amino, alkylamino, halo, nitro, cyano, thiol, thiol ether, amide, epoxy, aziridine, carboxylate, carboxylate ester (CO₂R⁴⁰), sulphoxide (OSO₂R^{4e}) guinadine, acyl, imidazole, indole, optionally substituted phenyl, alkoxy, aryloxy, acyloxy and acyl amino;
R⁴⁰ is C₁₋₄ alkyl or optionally substituted phenyl.

25. A compound according to claim 24 in which B¹ is XIV 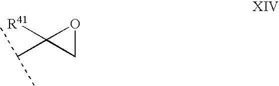
wherein R⁴¹ is selected from the group consisting of H, optionally substituted C₁₋₄ alkyl, C₁₋₄ alkoxy, optionally substituted phenyl, C₇₋₁₂ aralkyl, optionally substituted heteroaryl and a ligand.

26. A compound according to claim 25 in which B¹ is XV 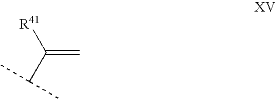
wherein R⁴¹ is defined as in claim 25.

27. A compound according to claim 25 in which R⁴¹ is CH₃.

28. A compound according to claim 24 in which R³¹ is optionally substituted naphthyl.

29. A compound according to claim 28 in which R³¹ is III

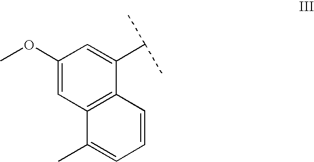

30. A compound according to claim 24 in which X⁴ is O.

31. A compound according to claim 24 in which R³² and R³³, together with the nitrogen to which they are attached form a ring of formula XIII.

32. A compound according claim 31 in which R³⁴ is CH₂A⁴ and R³⁵ is H.

33. A compound according to claim 32 in which t is 2 and R³⁷ is CH₂A⁴, in which A⁴ is the same as A⁴ in R³⁴.

34. A compound according to claim 24 in which Y¹ is NH.

35. A compound according to claim 24 in which Z¹ is (CH₂)₂.

36. A compound according to claim 24 which is

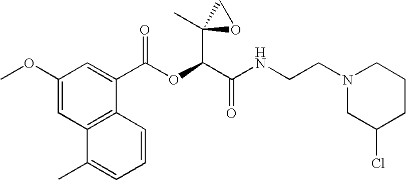

37. A compound according to claim 24 for use in a method of treatment of an animal by therapy.

38. Use of a compound according to claim 24 in the manufacture of a composition for use in a method of treatment of an animal, preferably in anti-tumour treatment.

39. A pharmaceutical composition comprising the compound of claim 24 and a pharmaceutically acceptable excipient.

40. A synthetic method in which a compound of formula XVI 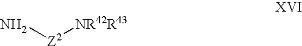
wherein Z² is C₁₋₇ alkanediyl;
R⁴² is (CH₂)₂A⁵ and R⁴³ is H or the same group as R⁴², or R⁴² and R⁴³ together with the nitrogen to which they are attached form a ring of formula XVII 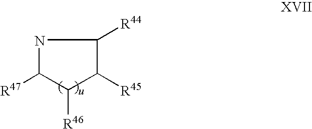
in which R⁴⁴ is CH₂A⁵ and R⁴⁵ is H or R⁴⁴ is H and R⁴⁵ is A⁵;
R⁴⁷ is H or the same group as R⁴⁴ and the or each R⁴⁶ is H or the same group as R⁴⁵
u is 1-4;
A⁵ is a leaving group, hydroxyl, protected hydroxyl or a halogen atom;
is reacted with a compound of formula XVIII 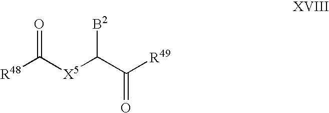
wherein R⁴⁹ is selected from the group consisting of a leaving group or a halogen;
in which X⁵ is selected from the group consisting of O, S and NR⁴⁶ in which R⁴⁶ is H or C₁₋₄ alkyl or is linked to B₂;
R⁴⁸ is optionally substituted phenyl, optionally substituted naphthyl, anthranyl or optionally substituted heteroaryl;
B² is selected from the same group as B¹ with the proviso that the substitutent groups may be protected;
such that R⁴⁹ is replaced by XIX

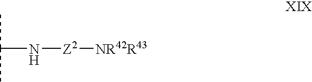

41. A method according to claim 40 in which at least one group A⁵ is hydroxyl or protected hydroxyl and in which the product is reacted with a halogenating compound optionally after deprotection to replace the or each A⁵ hydroxyl group by a halogen atom.

42. A method according to claim 41 in which the halogenating agent is a chlorinating agent.
